(19) United States
(12) Patent Application Publication
(10) Pub. No.: US 2009/0227935 A1
(43) Pub. Date: Sep. 10, 2009

(54) DRESSING SUPPORT

(76) Inventors: Andrea Zanella, Mirandola (IT); Rosanna Toscano, Parabiago (IT)

Correspondence Address: NIXON & VANDERHYE, PC, 901 NORTH GLEBE ROAD, 11TH FLOOR, ARLINGTON, VA, 22203, US

(21) Appl. No.: 12/158,890

(22) PCT Filed: Dec. 18, 2006
(86) PCT No.: PCT/EP2006/069819

§ 371 Date: Sep. 25, 2008

(30) Foreign Application Priority Data
Dec. 23, 2005 (IT) MO2005A000343

(51) Int. Cl. A61N 1/36; A61F 13/02; A61L 15/44

(52) U.S. Cl. 604/20; 607/50; 602/54; 602/56

(57) ABSTRACT

A dressing support comprises a supporting element arranged for adhering in a removable manner to a skin portion of a patient, a dressing element, an electrode arrangement, the dressing element and the electrode arrangement being arranged in the supporting element in such a way as to be able to come into contact in use with the skin portion, and a window obtained in the supporting element near the dressing element.

### CROSS-REFERENCE TO RELATED APPLICATIONS

This application is the U.S. national phase of International Application No. PCT/EP2006/069819, filed 18 Dec. 2006, which designated the U.S. and claims priority to Italy Patent Application No. M02005A000343, filed 23 Dec. 2005, the entire contents of each of which are hereby incorporated by reference.

### DESCRIPTION

The invention relates to a dressing support, in particular usable for treating vascular and/or diabetic bedsores.

In the medical field, the so-called advanced dressings are known, namely adhesive dressing supports comprising pharmacologically active substances that can be applied to a portion of skin surface of a patient for a preset period of time so as to release the aforesaid substances to the patient slowly. In particular, advanced dressings are known that are usable for treating bedsores and contain hydrocolloids, which are able to absorb the exudates produced by the bedsore, and substances, such as collagen, which act as tissue regenerators.

In addition to the use of advanced dressings, a treatment of the vascular and/or diabetic bedsore has been devised that is based on administering a specific vasoactive electric stimulation, which is obtainable by means of an electrostimulating apparatus disclosed in WO02/09809. This electric stimulation is able to cause vasodilation and release of VEGF (Vascular Endothelial Growth Factor), i.e. an endogenous growth factor that, being involved in the regeneration and proliferation of the blood vessels, may positively influence the progress of the vascular and/or diabetic bedsore.

Combining a pharmacological treatment, i.e. the advanced dressing, with an electrostimulating treatment, would thus enable to intervene in an effective manner on a pathology occurring with significant frequency in aged patients forced to be immobile.

Currently, however, it is not possible to treat a patient, which is affected by a vascular and/or diabetic bedsore, by using simultaneously an advanced dressing and the aforesaid electric stimulation. In fact, in order to be able to conduct the latter, it is necessary to apply to a skin region of the patient surrounding the vascular and/or diabetic bedsore a certain number of electrodes connected to the aforesaid electrostimulating apparatus. This means that if the bedsore is treated with an advanced dressing the latter has to be removed to enable the electrodes to be suitably positioned. Once the electrostimulation has been performed, the electrodes can be removed and a new advanced dressing can be applied at the vascular and/or diabetic bedsore.

A drawback arising from the situation disclosed above consists in the fact that the patient cannot take advantage of the pharmacological treatment and the electrostimulation treatment simultaneously, as the latter of necessity have to be performed alternatively.

Another drawback consists in the fact that, when the advanced dressing is removed to make place for the electrodes, the pharmacological treatment is interrupted and resumes only after a new advanced dressing is applied to the skin of the patient.

This entails an undesired extension of treatment time and an increase in the consumption of disposable sanitary material, with a consequent increase in treatment costs.

A further drawback is due to the fact that the nursing staff has to intervene frequently on the patient in order to remove, and subsequently reapply, the advanced dressing.

This entails a higher number of visits to be made to the patient when the latter is a home patient, with consequent further increase in treatment costs.

Still another drawback of known advanced dressings consists of the need to have to remove completely, and therefore replace, an advanced dressing in order to be able to examine the state of a lesion. This involves an undesired increase in the consumption of sanitary material and a consequent increase in treatment costs.

FR 2766376 discloses a device comprising a dressing provided with a layer for absorbing the wound exudates and electric supply means to circulate a current in the wound, through the dressing. The layer consists of a dry hydrophile layer, which is substantially non-conductive at the time it is applied on the wound. The subsequent migration of the exudates into the thickness of the layer controls the activation of the electric supply means, concomitantly with the continuation of the process of exudate absorption by the layer.

GB 2406519 discloses a dressing for treating damaged tissue, comprising a pair of electrodes (e.g. carbon fibre), a conductive gel to ensure good electrical contact with the tissue and a conductive tissue gel, which is absorbed by the wound being treated to allow current to pass more easily between the electrodes. The tissue gel is a hydropolymer releasing oxygen into the tissue to enhance the cell regeneration process. The dressing further incorporates a control unit, consisting of a microcontroller and a battery, and a sensor for detecting an environmental parameter of the tissue (e.g. oxygen, pH, bacterial infection, temperature).

US 2004/077984 discloses a film dressing for catheter sites, having a foam layer, to which a moisture vapor semi-permeable film may be secured. The moisture vapor semi-permeable film may be coated with an adhesive for application to the catheter site, which adhesive may vary in strength and pattern. A window in the foam layer allows for viewing of the catheter site. The dressing may further have a silicone-coated liner for protecting the semi-permeable film.

EP 0541251 discloses a dressing, comprising a pad contacting the wound without adhering thereto and absorbing wound exudate, a substrate adhered to the pad which is impermeable to both bacteria and liquid water and allows the extent of wound exudate absorption by the pad to be observed therethrough, and a cover sheet adhered to the substrate and having an opening therethrough. The substrate and pad are dimensioned such that they extend beyond the dimensions of the cover opening while allowing a portion of adhesive on the cover sheet to remain uncovered to permit the dressing to be adhered to a patient's skin around the wound.

US 2002/0169405 discloses a self adherent dermal wound window dressing, including a fabric tape layer having an adhesive skin adhering side, an opposite non-adhesive side and an opening therein to allow viewing therethrough. The opening is closed by a semipermeable transparent film layer, having an adhesive skin adhering side and an opposite non-adhesive side. The film layer non-adhesive side is adhered on the adhesive side of the fabric layer around the opening such that the fabric layer extends beyond the periphery of the transparent film layer. An absorbent fiber layer having an opening generally corresponding to the opening in the fabric tape layer, is mounted on the adhesive skin adhering side of the transparent film layer such that the openings in the absorbent fiber and fabric tape layers are in alignment and the transparent film layer extends beyond the periphery of the absorbent fiber layer.

U.S. Pat. No. 6,124,520 discloses a self adherent window dressing including a fabric tape layer having an adhesive side, an opposite non-adhesive side and an opening therein to allow viewing of a wound or injection site therethrough. A semipermeable transparent film layer closes the opening in the fabric tape layer and has an adhesive side and an opposite non-adhesive side. The film layer adhesive side is adhered on the non-adhesive side of the fabric layer around the opening. An absorbent fiber layer having an opening corresponding to the opening in the fabric tape layer is mounted on the adhesive side of the fabric tape layer such that the openings in the absorbent fiber and fabric tape layers are in alignment and the fabric tape layer extends beyond the periphery of the absorbent fiber layer. A non-adherent porous film layer of a shape generally corresponding to the shape of the absorbent layer is adhered to the fiber layer.

WO 2004/110253 discloses a device for preventing or treating ulcers, including an absorbent layer and an upper layer which may be transparent or opaque, and is preferably curved downward to engage the absorbent layer and minimize contact to the ulcer. A sensor or signaling member is provided for outputting an electrical signal in response to a flexible deformation of the upper layer indicating an excessive pressure. A warning member (light or alarm) replies to the signaling member for notifying the patient, or caregiver, of excessive pressure.

An object of the invention is to improve advanced dressings of known type.

Another object is to improve the advanced dressings for vascular and/or diabetic bedsores.

A further object is to provide an adhesive support for an advanced dressing that can be kept adhering to a skin lesion whilst the latter receives a concomitant electrostimulation treatment.

Still a further object is to provide an adhesive support for an advanced dressing that enables a lesion to be inspected without the adhesive support having to be completely removed.

In a first aspect of the invention, there is provided a dressing support, comprising a supporting element, arranged for adhering in a removable manner to a skin portion of a patient, a dressing element and an electrode arrangement, said dressing element and said electrode arrangement being arranged in said supporting element in such a way as to be able to come into contact in use with said skin portion, wherein said dressing support further comprises a window obtained in said supporting element near said dressing element.

Owing to this aspect of the invention, an advanced dressing is made available that is provided with electrodes, said dressing being applicable to a portion of skin surface which is affected, for example, by a vascular and/or diabetic bedsore. This advanced dressing can be connected to an electrostimulating apparatus, so as to enable a user to conduct local electrostimulation that acts synergically with the substances contained in the advanced dressing.

The advanced dressing can thus be left in the zone when the electrostimulation is conducted, which enables pharmacological therapeutical treatment not to be interrupted and time and money to be saved in a significant manner.

Further, it is possible to avoid, or at least reduce, home visits by nursing staff, inasmuch as it is no longer necessary to remove the advanced dressing before performing the electrostimulation and then apply the dressing again after performing the electrostimulation.

In one embodiment, a dressing element made of an absorbent material is provided, which is particularly useful if the advanced dressing has to be applied to a “weeping” vascular and/or diabetic bedsore, i.e. a secreting bedsore.

In another embodiment, the electrodes are connected together so as to form a circuit comprised in the supporting element. In this way, it is possible to use a single cable to connect the electrodes with the electrostimulating apparatus.

In a further embodiment, the supporting element comprises electrodes marked by numbers. In this way, a user who performs a number of consecutive electrostimulations can rotate the dressing each time so as to apply each electrode to mutually adjacent zones of the same bodily region.

In second aspect of the invention, a dressing support is provided, comprising a supporting element, arranged for adhering in a removable manner to a skin portion of a patient, a dressing element, arranged in said supporting element in such a way as to be able to come into contact in use with said skin portion, and a window obtained in said supporting element near said dressing element.

Owing to this aspect of the invention, an advanced dressing is made available that enables a user to remove the dressing element and inspect a lesion without having to remove the adhesive support completely.

It is further possible to replace the dressing element, leaving the respective adhesive support in place.

This enables the consumption of disposable sanitary material to be reduced significantly and therefore enables treatment costs to be reduced substantially.

In one embodiment, the supporting element comprises a movable flap, which is shaped and positioned in such a way as to close the window. The movable flap is foldable and is thus alternatively openable and/or closable.

In another embodiment, the movable flap is completely removable, and is replaced after each opening of the window.

The invention will be better understood and carried into effect with reference to the attached drawings, in which some embodiments of the invention are shown by way of non-limiting example, in which:

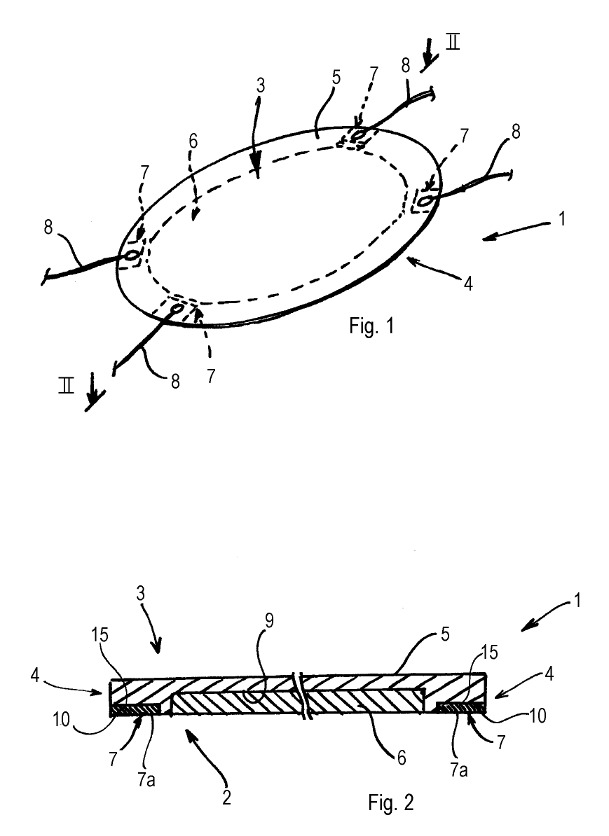

FIG. 1 is an incomplete and fragmentary schematic perspective view, showing an advanced dressing provided with electrodes;

FIG. 2 is an enlarged, incomplete and fragmentary cross section taken along the plane II-II in FIG. 1;

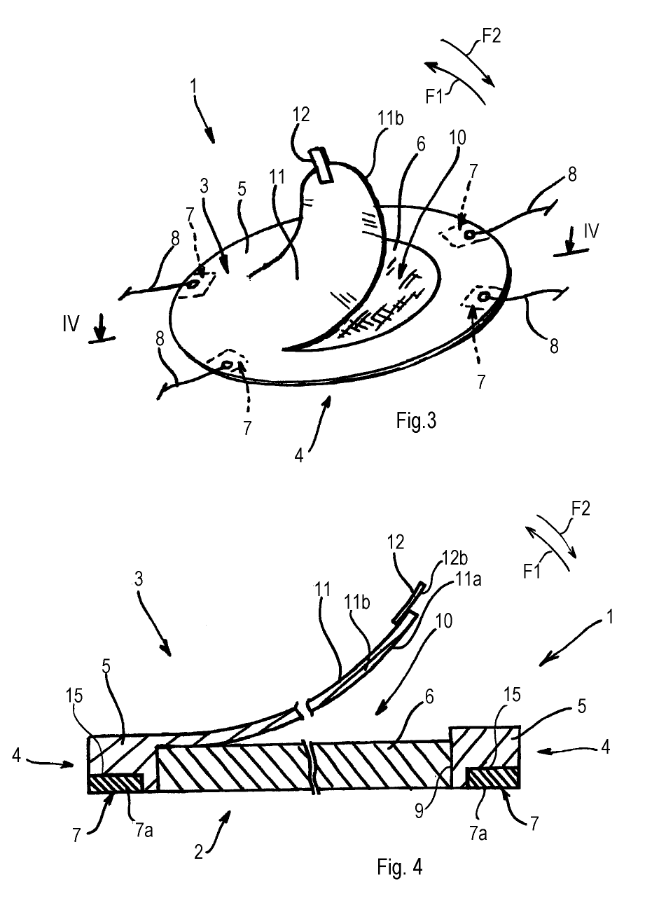

FIG. 3 is an incomplete and fragmentary schematic perspective view, showing an embodiment of the advanced dressing in FIG. 1 provided with a movable flap;

FIG. 4 is an enlarged, incomplete and fragmentary cross section taken along the plane III-III in FIG. 3;

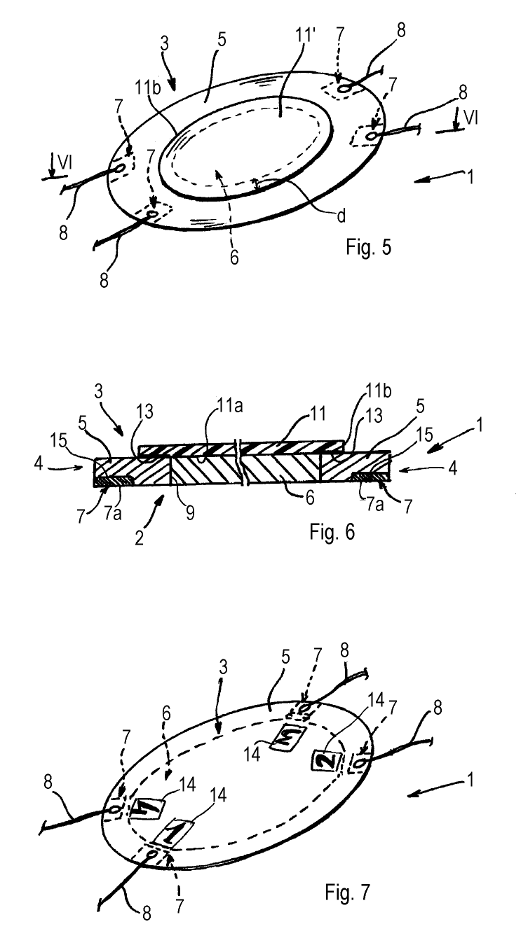

FIG. 5 is an incomplete and fragmentary schematic perspective view, showing another embodiment of the advanced dressing in FIG. 1 provided with a removable flap;

FIG. 6 is an enlarged incomplete and fragmentary cross section, taken along the plane VI-VI in FIG. 5;

FIG. 7 is an incomplete and fragmentary schematic perspective view, showing a further embodiment of the advanced dressing in FIG. 1 in which the electrodes are marked by means of numbers.

With reference to FIGS. 1 and 2, a dressing support 1, for example disc-shaped, comprises a supporting element 5, a dressing element 6 and four electrodes 7 for electrostimulation of known type.

The supporting element 5 is made of flexible material and comprises a contact face 2 and a visible face 3, which are mutually opposite and separated by an edge portion 4. In use, when the dressing support 1 is applied to a portion of skin surface of a patient (not shown) so as to cover a lesion, such as for example a vascular and/or diabetic bedsore, the contact face 2 is juxtaposed to the skin surface, and therefore to the lesion, whilst the visible face 3 is opposite the portion of skin surface. The supporting element 5 consists of a transparent hydrophilic membrane, for example a polyurethane membrane. The latter is permeable to the serous exudate produced by the lesion, which can thus pass to the external environment whilst microorganisms and/or particles of different kinds remain blocked outside the membrane.

In the contact face 2 of the supporting element 5 a seat 9 is obtained that is circle-shaped and inside which there is inserted the dressing element 6, which is shapingly coupled with the seat 9. The seat 9 is obtained in a substantially central portion of the contact face 2. The dressing element 6 (shown in FIG. 1 by means of a dotted line) consists of a flexible membrane in polyurethane, or in another biocompatible material, and is impregnated with substances acting as tissue regenerators, for example collagen, which are released slowly to the lesion.

In a peripheral portion of the contact face 2, said peripheral portion being comprised between the dressing element 6 and the edge portion 4 of the supporting element 5, an adhesive layer is made that is of known type and is not shown, by means of which the contact face 2, and therefore the dressing support 1, is kept adhering to the skin of the patient.

Each electrode 7 (shown, in FIG. 1, by means of a dotted line) is received in a corresponding groove 15 obtained in the contact face 2 near the edge portion 4. The groove 15 is made in such a way as to contain the electrode 7 completely and to enable a contact surface 7a of the electrode 7 to enter into contact, in use, with the skin of the patient. Each electrode 7 is further provided with an electric connecting cable 8 by means of which it can be connected to an electrostimulating apparatus (not shown) like the one disclosed in WO02/09809.

The support 1 disclosed above is particularly suitable for treating “dry”, i.e. non-secreting, vascular and/or diabetic bedsores.

In an embodiment that is not shown, which is particularly indicated for treating “weeping”, i.e. secreting, vascular and/or diabetic bedsores, there is provided an embodiment of the dressing element 6 that is made in absorbent material, for example polyurethane sponge.

In another embodiment that is not shown, the four electrodes 7 are mutually connected so as to form a circuit comprised inside the supporting element 5. In this way it is possible to connect the electrodes to the electrostimulating apparatus by means of a single connecting cable, which makes the dressing support 1 more convenient in use and simplifies the structure thereof.

In a further embodiment that is not shown, the supporting element 5 is made in the shape of a quadrilateral.

In a still further embodiment that is not shown, the dressing element 6 is made in the shape of a quadrilateral and is received in a seat 9 that is correspondingly made in the shape of a quadrilateral.

With reference to FIGS. 3 and 4, there is provided an embodiment of the dressing support 1 in which there is provided a foldable flap 11 in the visible face 3 of the supporting element 5. The foldable flap 11 is obtained during the manufacture of the support 1 by means of an approximately semicircular cut, which is made at the visible face 3 with known devices and methods. The foldable flap 11 is defined by a free margin 11b, approximately made in the shape of a half-ring, which is interrupted where the foldable flap 11 is continued with the surrounding visible face 3. The shape and dimensions of the foldable flap 11 correspond substantially to the shape and dimensions of the adjacent seat 9, and therefore of the dressing element 6 contained in the latter. The foldable flap 11 is provided with a grip portion 12, which is strip-shaped and protrudes from the free margin 11b and can be grasped and pulled by a user so as to raise the foldable flap 11 in a direction indicated by the arrow F1. In this way, the user opens an inspection window 10, having shape and dimensions substantially corresponding to those of the foldable flap 11, of the seat 9 and of the dressing element 6. The user can thus remove the dressing element 6 and inspect the lesion, as well as replacing the dressing element 6 if necessary. After inspecting the lesion and/or after replacing the dressing element 6, the user can close the inspection window 10 by moving the foldable flap 11 toward the seat 9 in a direction indicated by the arrow F2, which direction is opposite the direction F1.

The grip portion 12 comprises an adhesive face 12b, which faces the visible face 3 and by means of which the foldable flap 11 can be anchored in a removable manner to the visible face 3. In this way, the foldable flap 11 cannot be lifted accidentally and the inspection window 10 is kept shut.

The embodiment of the support 1 provided with the foldable flap 11 disclosed above can be used with the dressing element 6 made in polyurethane sponge disclosed above for treating secreting vascular and/or diabetic bedsores.

In an embodiment that is not shown, the foldable flap 11 is devoid of the grip portion 12, and the inspection window 10 can be kept shut by using a strip of sticking plaster of known type.

In another embodiment that is not shown, the foldable flap 11 is made in the shape of a quadrilateral and, correspondingly, the inspection window 10, the seat 9 and the dressing element 6 are quadrilateral-shaped.

With reference to FIGS. 5 and 6, there is provided another embodiment of the dressing support 1 comprising a removable flap 11′. The latter is circle-shaped and can be removed completely from the visible face 3, whilst the seat 9 extends throughout the thickness of the supporting element 5. The removable flap 11′ has a greater diameter than the diameter of the inspection window 10, which is circle-shaped, and peripherally overlaps the dressing element 6 by a distance d, which is comprised between the free margin 11b and the inspection window 10. In this way, in the removable flap 11′ there is defined a peripheral anchoring portion 13, which is ring-shaped and is provided, on a face thereof adjacent to the visible face 3, with an adhesive layer (not shown) of known type. The removable flap 11′ is thus maintained adhering to the supporting element 5 and in turn keeps the inspection window 10 closed. In use, a user can grasp the removable flap 11′ at the peripheral anchoring portion 13 and pull the latter so as to remove the removable flap 11′ and open the inspection window 10. After inspecting the lesion and/or replacing the dressing element 6, the user can juxtapose a new removable flap 11′ to the supporting element 5 so as to make the adhesive layer of the peripheral anchoring portion 13 adhere to an adjacent portion of visible face 3.

In an embodiment that is not shown, the removable flap 11′ is made in the form of a quadrilateral and, correspondingly, the inspection window 10, the seat 9 and the dressing element 6 are quadrilateral-shaped.

In another embodiment that is not shown, the removable flap 11′ is provided with a grip portion.

With reference to FIG. 7, there is provided a further embodiment of the dressing support 1 in which, on the visible face 3, there are printed identification numbers 14 near the electrodes 7. Owing to the identification numbers 14, a user can suitably orientate the dressing support 1 and choose where to position the electrodes 7. In this way, the user can perform electrostimulation, and produce a consequent vasoactive effect, in a plurality of zones comprised in the same bodily region.

1. Dressing support, comprising supporting means (5) arranged for adhering in a removable manner to a skin portion of a patient, dressing means (6) and electrode means (7), said dressing means (6) and said electrode means (7) being arranged in said supporting means (5) in such a way as to be able to come into contact, in use, with said skin portion, characterised in that said dressing support further comprises window means (10) obtained in said supporting means (5) near said dressing means (6).

2. Dressing support according to claim 1, wherein said supporting means (5) comprises a supporting element (5) made in the form of flexible membrane.

3. Dressing support according to claim 2, wherein said supporting element (5) comprises a visible face (3) and a contact face (2) that are mutually opposite, said contact face (2) being arranged for adhering in a removable manner, in use, to said skin portion.

4. Dressing support according to any preceding claim, wherein said dressing means (6) comprises a dressing element (6) made in, the form of flexible membrane.

5. Dressing support according to claim 4, as appended to claim 3, wherein said dressing element (6) is received in a shapingly coupled manner in seat means (9), said seat means (9) being obtained in said contact face (2) and being such as to enable said dressing element (6) to enter into contact, in use, with said skin portion.

6. Dressing support according to claim 5, wherein said seat means (9) is obtained in a central portion of said contact face (2).

7. Dressing support according to claim 5, or 6, wherein said seat means (9) communicates with said window means (10) near said visible face (3).

8. Dressing support according to claim 3, or to any one of claims 4 to 7 as appended to claim 3, wherein in said visible face (3) there is comprised flap means (11; 11′) arranged for alternatively opening and/or closing, in use, said window means (10).

9. Dressing support according to claim 8, wherein said flap means (11; 11′) comprises a foldable flap (11).

10. Dressing support according to claim 9, wherein said foldable flap (11) is provided with grip means (12) that is graspable, in use, so as to move said window means (10) toward, or away from, said foldable flap (11).

11. Dressing support according to claim 10, wherein said grip means (12) is strip-shaped and comprises an adhesive portion (12b), said adhesive portion (12b) being juxtaposable, in use, to said visible face (3).

12. Dressing support according to any one of claims 8 to 11, wherein said flap means (11; 11′) comprises a removable flap (11′).

13. Dressing support according to claim 12, wherein said removable flap (11′) comprises a peripheral anchoring portion (13), said peripheral anchoring portion (13) being juxtaposable, in use, to said visible face (3) and being provided with an adhesive layer.

14. Dressing support according to claim 12, or 13, wherein said removable flap (11′) is provided with grip means.

15. Dressing support according to claim 4, or to any one of claims 5 to 14 as appended to claim 4, wherein said dressing element (6) can be inserted into, or removed from, said seat means (9) through said window means (10).

16. Dressing support according to claim 4, or to any one of claims 5 to 15 as appended to claim 4, wherein said dressing element (6) is impregnated with a substance having a pharmacological action.

17. Dressing support according to claim 16, wherein said substance having pharmacological action comprises a tissue regenerator.

18. Dressing support according to claim 17, wherein said tissue regenerator comprises collagen.

19. Dressing support according to claim 4, or to any one of claims 5 to 18 as appended to claim 4, wherein said dressing element (6) is made of an absorbent material.

20. Dressing support according to claim 19, wherein said absorbent material comprises a polyurethane sponge.

21. Dressing support according to claim 2, or to any one of claims 3 to 20 as appended to claim 2, wherein said supporting element (5) is made in the form of transparent membrane.

22. Dressing support according to claim 21, wherein said transparent membrane is made of a hydrophilic material.

23. Dressing support according to claim 22, wherein said hydrophilic material comprises polyurethane.

24. Dressing support according to claim 3, or to any one of claims 4 to 23 as appended to claim 3, wherein said electrode means (7) comprises electrodes (7) for electrostimulation associated with said contact face (2).

25. Dressing support according to claim 24, wherein said electrodes (7) are inserted into groove means (15) obtained in said contact face (2), said groove means (15) being made in such a way as to enable a portion (7a) of said electrodes (7) to enter into contact, in use, with said skin portion.

26. Dressing support according to claim 24, or 25, wherein said electrodes (7) are associated with said contact face (2) so as to be in a peripheral position with respect to said dressing element (6).

27. Dressing support according to any one of claims 24 to 26, wherein said electrodes (7) are provided with corresponding supply cables (8).

28. Dressing support according to any one of claims 24 to 27, wherein said electrodes (7) are mutually connected so as to form a circuit provided with a single supply cable.

29. Dressing support according to any one of claims 24 to 28, as appended to claim 3 or as appended to any one of claims 4 to 23 as appended to claim 3, wherein said electrodes (7) are marked by reference number means (14) obtained in said visible face (3), said reference number means (14) being arranged for enabling said electrodes (7) to be orientated, in use, with respect to said skin portion.

30. Dressing support according to any one of claims 24 to 29, wherein four of said electrodes (7) are provided.

31. Dressing support according to any one of claims 3 to 30, wherein said contact face (2) is provided with adhesive layer means.

32. Dressing support according to claim 31, as appended to claim 3 or as appended to any one of claims 4 to 30 as appended to claim 3, wherein said adhesive layer means is made in a portion of said contact face (2) comprised between said dressing means (6) and said electrode means (7).

33. Dressing support according to claim 2, or to any one of claims 3 to 33 as appended to claim 2, wherein said supporting element (5) is disc-shaped.

34. Dressing support according to claim 2, or to any one of claims 3 to 33 as appended to claim 2, wherein said supporting element (5) is quadrilateral-shaped.

35. Dressing support according to any preceding claim, wherein said window means (10) comprises a circular inspection window (10).

36. Dressing support according to any preceding claim, wherein said window means (10) comprises a quadrilateral-shaped window (10).

37. Use of a dressing support according to any one of claims 1 to 36 for treating a bedsore.

38. Use of a dressing support according to any one of claims 1 to 36 for treating a vascular and/or diabetic bedsore.
